(19) United States
(12) Patent Application Publication
(10) Pub. No.: US 2023/0121265 A1
(43) Pub. Date: Apr. 20, 2023

(54) REMOTE DIAGNOSTIC TESTING SYSTEMS AND METHODS

(71) Applicant: EMED LABS, LLC, Miami, FL (US)

(72) Inventors: Zachary Carl Nienstedt, Wilton Manors, FL (US); Maria Teresa Pugliese, Miami, FL (US); Adam Charles Carlson, Miami, FL (US); Samuel A. Miller, Hollywood, FL (US); Marco Magistri, Miami, FL (US); Nicholas Atkinson Kramer, Seattle, WA (US)

(21) Appl. No.: 18/047,518

(22) Filed: Oct. 18, 2022

Related U.S. Application Data
(60) Provisional application No. 63/257,445, filed on Oct. 19, 2021.
(60) Provisional application No. 63/257,986, filed on Oct. 20, 2021.
(60) Provisional application No. 63/262,792, filed on Oct. 20, 2021.
(60) Provisional application No. 63/271,666, filed on Oct. 25, 2021.
(60) Provisional application No. 63/268,675, filed on Feb. 28, 2022.

(51) Int. Cl. G16H 40/67; G16H 30/40; G16H 80/00; G06T 7/80

(52) U.S. Cl. CPC G16H 40/67; G06T 7/80; G16H 30/40; G16H 80/00; G06T 2207/30168

(57) ABSTRACT

As one example of remote health testing or diagnostics using a personal device and remote image quality verification, a user may be prompted to take an image, for example, of a reference card, using a camera of his or her personal user device. The image of the reference card can be uploaded by the personal user device to a server on the cloud. The server can be configured to evaluate the image to determine whether the image is already of sufficient quality (for being used in medical diagnostics) or the image can be processed using designated processing techniques and parameters to produce a processed image of sufficient quality. Finally, the server can respond to the user device with an indication of the determination result.

### PRIORITY APPLICATIONS

Any and all applications for which a foreign or domestic priority claim is identified in the Application Data Sheet as filed with the present application are hereby incorporated by reference under 37 CFR 1.57.

This application claims the benefit under 35 U.S.C. § 119(e) of U.S. Provisional Patent Application No. 63/257,445, filed Oct. 19, 2021, U.S. Provisional Pat. Application No. 63/257,986, filed Oct. 20, 2021, U.S. Provisional Pat. Application No. 63/262,792, filed Oct. 20, 2021, U.S. Provisional Pat. Application No. 63/271,666, filed Oct. 25, 2021, and U.S. Provisional Pat. Application No. 63/268,675, filed Feb. 28, 2022, the contents of each of which are incorporated by reference herein.

### BACKGROUND

### Field

The present application is directed to systems, methods, and devices that are configured to enable or facilitate remote healthcare, such as for remote medical testing and diagnostics. Some embodiments provide for remote health care using a personal user device, such as a smartphone or tablet.

The present application also is directed to systems, methods, and devices that are configured to enable or facilitate image verification and/or processing, such as image verification and/or processing used during remote medical testing and diagnostics. Some embodiments provide for remote health care using a personal user device, such as a smartphone or tablet.

The present application also is directed to remote testing sessions, such as those including remote health testing or diagnostics. Some embodiments are directed at improving efficiency for remote testing sessions.

### Description

Use of telehealth to deliver healthcare services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the use of a user or patient’s personal user device, such as a smartphone, tablet, laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user’s convenience. However, remote or at-home testing introduces various additional logistical and technical issues, such as guaranteeing timely test delivery to a testing user, providing test delivery from a testing user to an appropriate lab, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

Remote or at-home healthcare often involves the use of a user or patient’s personal user device, such as the user’s smartphone, tablet, laptop, personal computer, or other device. While this greatly increases the availability and ease of access to healthcare, use of or reliance on the user’s personal user device may pose challenges. For instance, in many countries or regions, governmental regulatory bodies may provide strict requirements related to which devices can be used for medical testing or diagnostics. For example, in the United States, the Food and Drug Administration (FDA) must approve, in various circumstances, medical devices before they can be used for medical testing or diagnostics. Other regulatory bodies impose similar requirements throughout the world. Users’ personal user devices, such as smartphones, tablets, laptops, personal computers, or other devices, are often not approved for use as medical devices under these regulatory schemes. This can impose a significant limitation (or even a bar) on the use of personal user devices in the remote or at-home healthcare context.

Some of the systems, methods, and devices described in this application can facilitate and enable the use of personal user devices, such as smartphones, tablets, laptops, personal computers, or other devices, for remote or at-home healthcare through the use of medical sensor subsystems, which can be included in the personal user devices. The medical sensor subsystems can be configured such that they comply with the necessary regulatory requirements (e.g., qualifying themselves as medical devices) and such that they can be readily integrated into various personal user devices (where, in some instances, the various personal user devices themselves or as a whole need not comply with the regulatory requirements). The medical sensor subsystems can further be configured for use in remote or at-home healthcare, for example, as part of a remote health testing or diagnostic process. The medical sensor subsystem can also be configured such that the computation associated with a medical test that is carried out on the personal user device itself (e.g., on a processor of the personal user device) is minimized or even eliminated. Instead, the computing can be mainly or entirely performed on a remote server or device, such as a device in the cloud. The remote server or device can also be configured to comply with the necessary regulatory requirements. In this way, remote health testing or diagnostics, which would normally be required to be performed on a regulatorily compliant device, can be performed on a personal user device that itself is not regulatorily certified, but which includes regulatorily certified medical sensor subsystems that can be in electronic communication with regulatorily certified remote servers or devices over a network, such as the internet.

Examples of such medical sensor subsystems can include, for example, camera or imaging sensor subsystems, microphone or other audio recording subsystems, inertial measurement subsystems, pressure sensor subsystems, or others.

In some embodiments, a camera or imaging sensor subsystem can include one or more of the following components, among others: a camera module (including one or more of a lens array, a filter, an image sensor, or an autofocus mechanism) and supporting hardware (such as, for example, an image signal processor (ISP), an autofocus driver, and/or other supporting circuit components or control associated with the camera).

In some embodiments, a microphone or other audio recording subsystem can include one or more of the following components, among others: microphone hardware, preprocessing integrated circuits (if necessary), a printed circuit board (PCB) layout for noise and/or function, and or a case opening or porting.

In some embodiments, an inertial measurement subsystem can include one or more of the following components, among others: IMU hardware (e.g. accelerometers, magnetometers, and/or gyrometers or gyroscopes), preprocessing integrated circuits (if necessary), a printed circuit board (PCB) layout for noise and/or function, and or orientation and mounting structures.

In some embodiments, a pressure sensor subsystem can include one or more of the following components, among others: a pressure sensor, preprocessing integrated circuits (if necessary), a printed circuit board (PCB) layout for noise and/or function, and or a case opening or porting.

Some systems, methods, and devices described in this application can facilitate and enable the use of personal user devices, such as smartphones, tablets, laptops, personal computers, or other devices, for remote or at-home healthcare through the use of remote image quality verification. As described herein, with remote image quality verification, images captured during a health test or diagnostic with a camera of a personal user devices (e.g., the user’s own smartphone, tablet, laptop, personal computer, or other similar device) can be verified on a system or device that is remote (e.g., separate from) the user’s personal device. In some embodiments, the system of device that performs the remote image quality verification is located on a cloud-based network.

In some embodiments, the system or device that performs the remote image quality verification can be configured such that it complies with and/or receives approval from one or more of the regulatory bodies that regulate medical devices. By having such a system or device perform the image quality verification, in some instances, the personal user device itself need not receive regulatory approval. For instance, during a medical diagnostic test, a user can capture an image using a camera on his or her smartphone. The camera and/or the smartphone may not qualify as a medical device under the regulatory schemes. However, the image can be transmitted, for example, over a computer network such as the internet, to a remote system or device that, as described herein, can be configured to perform image quality verification. This system or device can be configured to qualify as a medical device under one or more regulatory schemes. By having such a device perform image quality verification, it can be possible to use the image captured by the user’s smartphone (which may not be a regulatorily approved device), but which image has been verified by a remote system or device (which can be a regulatorily approved device) for medical treatment, diagnosis, etc. This can greatly expand access to digital healthcare and provide improved patient outcomes, reliability, and accuracy.

Further, personal user devices, such as smartphones and tablets, are available in a wide variety of models from a number of different manufacturers. Many manufacturers provide new or updated models of their personal user devices on a yearly (or even more frequent) basis. Due to the wide variation in personal user devices, it can be difficult to ensure that images captured by such devices will be of sufficient quality for use in medical diagnosis or procedures. However, the remote image quality verification processes and techniques described here can provide a solution, verifying such images before they can be relied on for medical purposes.

As one example of remote health testing or diagnostics using a personal device and remote image quality verification, a user may be prompted to take an image, for example, of a reference card, using a camera of his or her personal user device. The image of the reference card can be uploaded by the personal user device to a server on the cloud. The server can be configured to evaluate the image to determine whether the image is already of sufficient quality (for being used in medical diagnostics under FDA or other regulatory requirements) or the image can be processed using designated processing techniques and parameters to produce a processed image of sufficient quality. Finally, the server can respond to the user device with an indication of the determination result.

In some remote testing implementations, users can be monitored during a testing procedure by a live proctor. Monitoring by a live proctor can help to ensure that test procedures are followed, verify identity, interpret test results, and so forth. However, users may encounter difficulties and delays if they have to take tests under the observation of a live proctor. High testing volumes can require many proctors and user experiences may suffer if they have to wait for a proctor to become available.

Some of the systems, methods, and devices described herein are directed to self-guided testing. These systems, methods, and devices may decrease the need for live proctors and/or may reduce the amount of time proctors must spend on each test, thereby reducing costs and improving efficiency. In some instances, users may be able to complete a test without real-time observation by a proctor. This can lead to improved user experiences as users do not have to wait for a proctor. In some embodiments, a proctor may be present for limited portions of a testing session. In some embodiments, a proctor may review a testing session, test result, etc., asynchronously.

Health testing and diagnostics often produce test results that may require interpretation. In some instances, result interpretation may require lab handling and can be time intensive and/or slow. Unfortunately, this can cause a delay in reporting medical diagnostic results to a patient or even create the possibility that the medical diagnostic test results become lost in transit during the process of delivery to the appropriate lab for interpretation. Remote or at home health testing and diagnostics can include tests which can be taken by a patient in his or her own home. In such cases, it may be desirable to have the results of the test interpreted in the patient’s own home, avoiding the need to send testing materials to a lab for interpretation of the results and the associated problems discussed above.

Some embodiments of this application can provide self-guided results interpretation that can be used to decrease or eliminate the time taken for results of a medical diagnostic test to be reported to the user or patient by, for example, having the user provide an initial interpretation of the test results along with sufficient information and data such that the user’s interpretation can later be reviewed and validated or rejected by a healthcare professional or testing proctor. In some embodiments, the user may capture and submit information (which can, for example, be image-based, video-based, audio-based, or text-based) regarding their medical diagnostic test results after the administration of a medical diagnostic test with the use of a user device, such as a smartphone, tablet, laptop, or personal computer. In some embodiments, with guidance, the user may also submit a self-attestation of their interpretation of the results of the medical diagnostic test.

After submission of the user’s interpretation of their results, in some embodiments, a proctor or other healthcare professional may interpret (e.g., verify or reject) the user’s interpretation of their medical diagnostic test results. At this point, the proctor may agree or disagree with the user’s self-attestation of the results. In some instances, if the proctor agrees with the user’s self-attestation of the medical diagnostic test results, then the result (e.g., an official result) may be issued. In other instances, if the proctor disagrees with the user’s self-attestation of the medical diagnostic test results, then an indication that the user’s selfreported interpretation of the test results is invalid may be issued. In some instances, if the proctor disagrees with the user’s interpretation of the results, the user may be provided with contact information for a follow up to discuss the interpretation of the results, for example, with a proctor or other healthcare professional. This can, for example, beneficially eliminate the need to transport the medical diagnostic test results to the appropriate lab for interpretation and/or eliminate the need for the user to wait for a remote proctor to become available at the time of testing to interpret the results in a live setting (e.g., through a live video conference) at the time of testing. This can provide an improved user experience in remote or at-home health testing and diagnostics.

In one aspect, a method for image analysis of a medical diagnostic test, the method comprises: receiving, by an image verification server, specifications of a camera of a user device; comparing, by the image verification server, the specifications to a database of specifications to determine if the specifications are sufficient for use in the medical diagnostic test; if the image verification server determines the specifications are sufficient for use in the medical diagnostic test, prompting, by a telehealth platform in communication with the image verification server, a user to capture an image of a reference using the camera of the user device; receiving, by the image verification server, the image of the reference; automatically processing, by the image verification server, the image of the reference using a calibration model to produce a processed image, wherein the calibration model is based on the specifications; determining, by the image verification server, whether a quality of the processed image is above a predetermined threshold; if the processed image does not have a quality above the predetermined threshold, automatically updating, by the image verification server, the calibration model, processing, by the image verification server, the processed image using the calibration model, and determining, by the image verification server, whether the quality of the processed image is above the predetermined threshold; if the processed image has a quality above the predetermined threshold, prompting, by the telehealth platform, the user to capture an image of a diagnostic test result; receiving, by the image verification server, the image of the diagnostic test result; processing, by the image verification server, the image of the diagnostic test result using the calibration model to produce a processed test image; transmitting, by the image verification server, the processed test image to the telehealth platform; and analyzing, by the telehealth platform, the processed test image to determine if the diagnostic test result is positive or negative.

The method may include one or more of the following features: (a) wherein the reference is a reference card comprising a unique code, graduated color strips, a plurality of color reference blocks, and a plurality of fiducials; (b) wherein the quality of the processed image is based on one or more of a resolution, a sharpness, a brightness, a noise, a dynamic range, a contrast, a saturation, and/or a white balance; (c) wherein the image verification server generates an image quality score of the quality of the processed image; (d) wherein the user device comprises a medical sensor subsystem, the medical sensor subsystem comprising: the camera; an image signal processor; an autofocus driver; and a circuit; (e)wherein the medical sensor subsystem meets guidelines or regulations of a regulatory body; (f) wherein the camera is separated from the image signal processor, the autofocus driver, and the circuit via a shield; (g) wherein the image verification server is configured to: receive a plurality of images from a plurality of user devices comprising the medical sensor subsystem; and update the calibration model based on the quality of the plurality of images; (h) wherein the image verification server is a remote image verification server separate from the user device; (i) wherein the image verification server transmits an indication of the determination whether the quality of the processed image is above the predetermined threshold to the user device and/or a partner device, wherein the partner device is a device of a proctor; and/or other features as described herein.

In another aspect, a method for self-guided testing can include: receiving, by a telehealth platform, a first video of a user performing one or more self-guided steps of a first phase of a medical diagnostic testing procedure, wherein the first video comprises a first frame rate; receiving, by the telehealth platform, a second video of the user performing one or more self-guided steps of a second phase of a of the medical diagnostic testing procedure, wherein the second video comprises a second frame rate, and wherein the second frame rate is higher than the first frame rate; transmitting, by the telehealth platform, the first video and the second video to a proctor device; displaying, by the telehealth platform, the first video and the second video to a proctor via the proctor device; receiving, by the telehealth platform, an input from the proctor, the input indicating whether the user performed the first phase and the second phase in compliance with the medical diagnostic testing procedure; based on the input from the proctor, selecting, by the telehealth platform, a third phase of the medical diagnostic testing procedure, wherein the third phase comprises one or more steps; and prompting, by the telehealth platform, the user to perform the one or more steps of the third phase.

The method may include one or more of the following features: (a) wherein the first phase comprises the user setting up testing materials or collecting a sample; (b) wherein the second phase comprises the user waiting for a predetermined amount of time; (c) wherein the input indicates the user performed the first phase and the second phase in compliance with the medical diagnostic testing procedure, and the one or more steps of the third phase comprise a test result interpretation procedure; (d) wherein the test result interpretation procedure comprises: receiving, by the telehealth platform, an image of a test result and an interpretation of the test result input by the user; displaying, by the telehealth platform, the image of the test result to the proctor via the proctor device; receiving, by the telehealth platform, a second interpretation of the test result input by the proctor; and analyzing, by the telehealth platform, the interpretation and the second interpretation to determine the test result of the medical diagnostic testing procedure, wherein said analyzing comprises comparing the interpretation and the second interpretation to determine if the interpretation and the second interpretation indicate a same test result; (e) wherein the input indicates the user performed the first phase and/or the second phase not in compliance with the medical diagnostic testing procedure, and the one or more steps of the third phase comprise the telehealth platform connecting the user with the proctor for assistance with the medical diagnostic procedure; and/or other features as described herein.

In another aspect, a method of interpreting test results of a medical diagnostic test can include: receiving, by a telehealth platform, an image of a test result and an interpretation of the test result input by a user; displaying, by the telehealth platform, the image of the test result to a proctor via a proctor device; receiving, by the telehealth platform, a second interpretation of the test result input by the proctor; and analyzing, by the telehealth platform, the interpretation and the second interpretation to determine a test outcome of the medical diagnostic test, wherein said analyzing comprises comparing the interpretation and the second interpretation to determine if the interpretation and the second interpretation indicate a same test result.

The method may include one or more of the following features: (a) wherein if the interpretation and the second interpretation indicate the same test result, the test outcome is the same test result; (b) wherein if the interpretation and the second interpretation do not indicate the same test result, the telehealth platform is configured to: analyze the image of the test result, via a results interpretation algorithm, to determine a third interpretation of the test result; display the image of the test result to a second proctor via a second proctor device; receive a fourth interpretation of the test result input by the second proctor; analyze the third interpretation and the fourth interpretation to determine the test outcome of the medical diagnostic testing procedure, by comparing the third interpretation and the fourth interpretation to determine if the third interpretation and the fourth interpretation indicate a same test result, if the third interpretation and the fourth interpretation indicate a same test result, the test outcome is the same result, and if the third interpretation and the fourth interpretation do not indicate a same test result, the test outcome is invalid, and the telehealth platform is configured to prompt the user to retake the medical diagnostic test; (c) wherein if the interpretation and the second interpretation do not indicate the same test result, the telehealth platform is configured to: connect the user to a representative, wherein the representative reviews the test result with the user to guide the user to modify the interpretation to the same test result as the second interpretation, and if the user modifies the interpretation to the same test result as the second interpretation, the test outcome is the same test result, if the user does not modify the interpretation to the same test result as the second interpretations, the representative indicates the test outcome is invalid; and/or other features as described herein.

For purposes of this summary, certain aspects, advantages, and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present application are described with reference to drawings of certain embodiments, which are intended to illustrate, but not to limit, the present disclosure. It is to be understood that the attached drawings are for the purpose of illustrating concepts disclosed in the present application and may not be to scale.

FIG. 1 is a diagram illustrating the use of a personal user device including medical sensor subsystems that are configured to communicate with a cloud compute resource to facilitate remote healthcare such as testing and diagnostics, according to an embodiment.

FIG. 2 is a diagram illustrating an embodiment of a camera subsystem comprising a camera module and supporting hardware.

FIG. 3 illustrates is a diagram illustrating the camera subsystem of FIG. 3 integrated into a personal user device illustrated a smartphone, according to an embodiment.

FIG. 4 is a diagram schematically illustrating additional components of the personal user device of FIG. 3 and shielding that can separate the camera subsystem from the other components, according to an embodiment.

FIGS. 5 and 6 provide examples of modern smartphone PCBs, showing subsystems arranged into shielded areas.

FIG. 7 illustrates an example process for remote image quality verification.

FIG. 8 illustrates another example process for remote image quality verification.

FIG. 9 illustrates an example process for image correction and verification.

FIG. 10 illustrates an example environment in which the remote image quality verification processes described herein can be implemented.

FIG. 11 provides details related to embodiments of reference cards.

FIGS. 12-14 illustrate an example embodiment of a user interface configured to guide a user through self-interpretation of the results of a remote or at home health or diagnostic test.

FIG. 15 illustrates an example embodiment of a proctor interface configured for use by a proctor in evaluating the user’s self-interpretation of the results of a remote-or at home health or diagnostic test.

FIG. 16 is a table illustrating example criteria which can be used for comparing a user’s and a proctor’s results interpretation of the results of a remote-or at home health or diagnostic test and resulting outcomes.

FIG. 17 is block diagram illustrating an embodiment of a protocol or method for self-guided results interpretation.

FIG. 18 is a block diagram illustrating an embodiment of a computer hardware system configured to run software for implementing one or more embodiments of the health testing and diagnostic systems, methods, and devices disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

### Medical Sensor Subsystems

As mentioned briefly above, some of the systems, methods, and devices described in this application can facilitate and enable the use of personal user devices, such as smartphones, tablets, laptops, personal computers, or other devices, for remote or at-home healthcare through the use of medical sensor subsystems, which can be included in the personal user devices. The medical sensor subsystems can be configured for use during remote healthcare such as testing and/or diagnostics. The medical sensor subsystems can be configured to minimize the computation performed on the personal user device itself, instead relying on one or more remote compute resources in communication with the medical sensor subsystems over a network, such as the internet. In some embodiments, the remote compute resource comprises a cloud compute resource. The remote compute resource can be configured to analyze data received from the medical sensor subsystem, provide test procedures or instructions for use, and/or provide results interpretation. In some embodiments, the medical sensor subsystems and the remote compute resources can be configured to comply with and/or be certified by the necessary regulatory bodies as a medical device, even if the personal user device itself does not.

In some embodiments, the systems, methods, and devices described in this application can facilitate and enable the use of personal user devices as a tricorder (e.g., recording (1) images and/or video, (2) audio, and (3) motion data). For example, a personal user device, such as a smartphone or other device can be configured to assist in the diagnosis of non-critical conditions, such as classifying skin conditions by analyzing phone camera images, classifying respiratory problems by analyzing phone microphone recordings of coughs, detecting snoring and sleep apnea by analyzing microphone and accelerometer data, and/or detecting gait abnormalities by analyzing accelerometer data. Although described as a tricorder providing three functions, other numbers of functions (e.g., one, two, three, four, five, or more) or other different combinations of functions can be used in some embodiments.

Additionally or alternatively, the personal user device can be configured to use its sensors to read the results of FDA-approved diagnostic tests (or diagnostic tests approved by similar regulatory agencies or governing bodies). In some embodiments, the results of some tests, such as some conventional tests, can be read using computer vision. Additionally or alternatively, the personal user device can be configured to interface to wearables that augment its ability to assist in medical monitoring and diagnosis. Additionally or alternatively, the personal user device can be configured to interface to other accessories that improve patient experience.

As described above, utilizing a user’s personal user device for anything beyond software as a medical device can encounter large regulatory barriers due to the disparate and consumer nature of personal user devices. Personal user devices, such as smartphones and tablets, are available in a wide variety of models from a number of different manufacturers. Further, many manufacturers provide new or updated models of their personal user devices on a yearly (or even more frequent) basis. Due to the wide variation in personal user devices, these devices are often not certified as medical devices by the various regulatory agencies. Still, there are many potential applications for using personal user device hardware to assist in diagnosis, reading of test results, etc., for example, in the context of remote healthcare. Thus, it would be desirable if such personal user devices could be reliably used as certified medical devices.

For a personal user device’s sensors to assist in diagnosis or to read diagnostic test results, it can be beneficial for the phone itself to act in the capacity of an approved medical device. However, it must be approved as such by the FDA (or CE or similar governing body worldwide). It is possible that a single model smartphone could get approved as a medical device in some capacity (e.g., using the camera to read test results). This approval, however, would be lengthy (likely 1 to 2 years minimum) and costly, and still would only apply to that specific device. The time required for the approval process on its own is a huge disincentive for phone manufacturers, as they want customers to upgrade as quickly as possible. In addition, in 2018 in the US, people waited only 24 months on average before upgrading to new devices. The fact that the approval applies to only a single phone model is a disincentive for developers of medical apps and other technologies that would require such an approval because the user base would be very small.

This application provides solutions to these challenges. Specifically, as described herein, this application proposes to create a system architecture that is safe and reliable and that meets FDA guidelines and regulations (or those of other regulatory bodies), while still being quick to adapt to different personal user device hardware and software architectures. This system architecture can include two major components. First, tightly controlled medical sensor subsystems that can provide medical device functionality can be developed that can be implemented on or incorporated into a wide range of personal user device hardware and software devices. Second, the system can be configured to minimize the computing done on the personal user device itself, favoring remote computing (e.g., in the cloud), thus providing that the medically significant decisions are made on the remote system and not on the device itself.

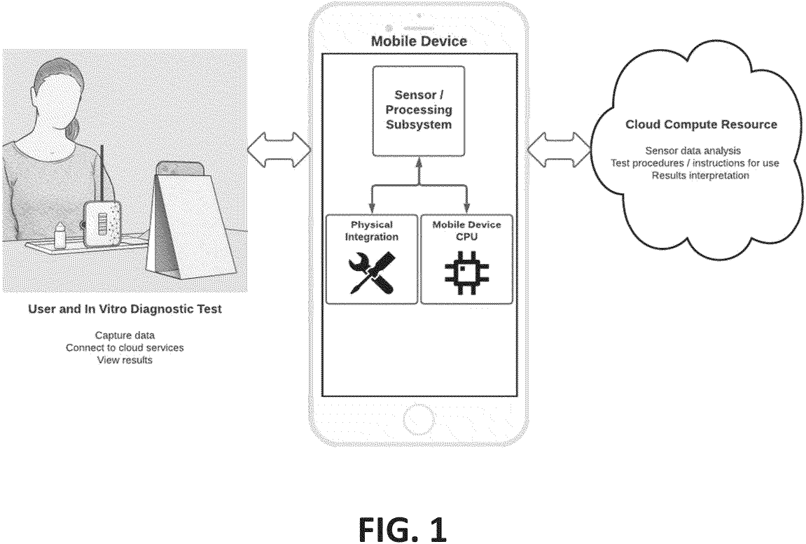

FIG. 1 provides an example. FIG. 1 is a diagram illustrating the use of a personal user device including medical sensor subsystems that are configured to communicate with a cloud compute resource to facilitate remote healthcare such as testing and diagnostics, according to an embodiment. As shown, a user utilizes a personal user device, such as the illustrated smartphone, to take a medical diagnostic test (left pane). In the illustrated example, the smartphone is positioned in a stand which orients the smartphone relative to the user and the other components of a test kit. For example, the smartphone can be oriented such that the user can see the display of the smartphone (on which can be displayed testing instructions, communications with a proctor, etc.) and a camera of the smartphone (e.g., a forward-facing camera has a view of the user and the test kit materials (such that a remote proctor or system can monitor the testing procedure).

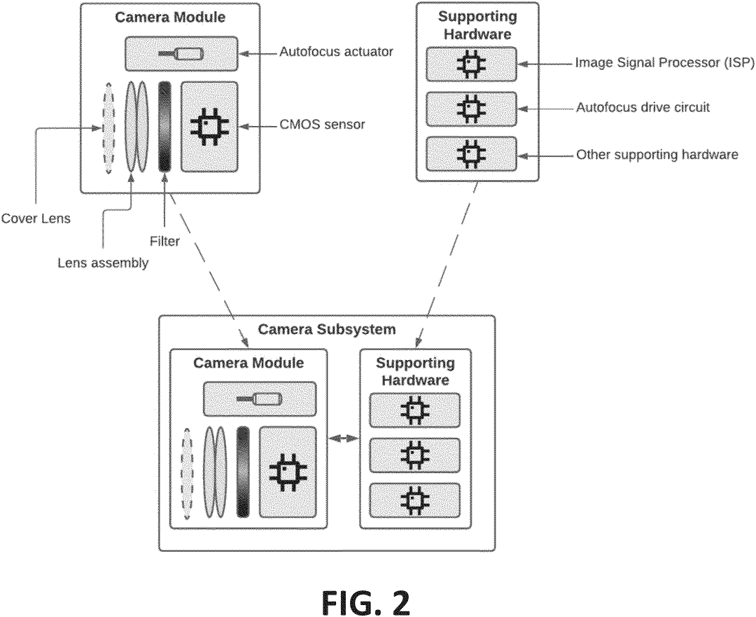

As shown schematically in the center pane, the smartphone can include a medical sensor subsystem (e.g., the illustrated “sensor / processing subsystem”) which can be physically integrated into the smartphone and in electronic communication with the mobile device CPU and/or other components of the smartphone. In the illustrated example, the medical sensor subsystem can include, for example, a camera subsystem such as the forward-facing camera that is used to monitor the user during the test. The medical sensor subsystem (in the illustrated example, the camera subsystem) can be configured as shown and described below with reference to FIGS. 2-4, for example. The medical sensor subsystem can be configured to provide the first major component of the system discussed above (e.g., tightly controlled medical sensor subsystems that can provide medical device functionality can be developed that can be implemented on or incorporated into a wide range of personal user device hardware and software device).

As illustrated in the right pane, the smartphone can be in communication with remote compute resource, such as the illustrated cloud compute resource. Communication between the smartphone and the remote compute resource can occur over one or more computer networks, including over the internet. The remote compute resource can be configured to work with the medical sensor subsystem in the smartphone to provide the second major component discussed above (e.g., minimizing the computation done on the personal user device itself, favoring remote compute (e.g., in the cloud), thus providing that the medically significant decisions are made on the remote compute resource and not on the device itself).

In the example of FIG. 1, as part of the test, the camera may be used to interpret results, for example, using computer vision of a test card or test strip. Images captured by the medical sensor subsystem (in this example, the camera subsystem) can be communicated to the remote compute resource for analysis. In this way, the medical diagnosis need not be performed by the processor of the mobile device itself.

The medical sensor subsystems can comprise specific combinations of hardware blocks and integration guidelines that can be approved for use as medical devices (e.g., by the necessary regulatory bodies) when installed in personal user device, such as smartphones, tablets, laptops, personal computers, or others. In some instances, all relevant components that make up the subsystems can be tightly controlled and treated as a single black box component that contains the subsystem. In some instances, a standardized connection and communication protocol with the personal user device’s main central processing unit (CPU) and other relevant components or subsystems can also be provided. For some instantiations, the method by which the subsystem is integrated into the personal user device may also be important. For example, this could include camera cover lenses, openings in the cover for microphones or pressure sensors, mounting considerations for inertial measurement components, etc. For some instantiations, the hardware and software functionality needed by a given sensor subsystem can be encapsulated in an ASIC (application-specific integrated circuit).

The medical sensor subsystems further are configured to transfer the computation and analysis of the results and/or other processing features from the personal user device to a remote server or device, such as a device located in the cloud. In this way, the personal user device itself does not run the software that actually produces results. It acts more as a client for data capture results review, with results being computed remotely (e.g., on the cloud). This can significantly reduce the risk and dependence on the mobile device operation and performance.

FIGS. 2-4 illustrate an example medical sensor subsystem configured as a camera subsystem for medical machine vision. In this example, the desired result is that any personal user device, regardless of brand, CPU, other peripherals, etc., can produce an identical image when equipped with the approved camera subsystem. “Identical” in this case can be defined as equivalent within acceptable variances of brightness, distortion, color reproduction, etc. In order to achieve the aforementioned goal, the following hardware can be included in the medical sensor subsystem as illustrated in FIG. 2. First, the subsystem can include a camera module which can include one or more of the following, among other components: a lens array, a filter, a sensor (such as an image sensor), an autofocus mechanism, a cover lens, and/or other components of the physical camera module itself. Second, the subsystem can include supporting hardware, such as an ISP (Image Signal Processor), an autofocus driver, and/or any other supporting circuit components or control associated with the camera.

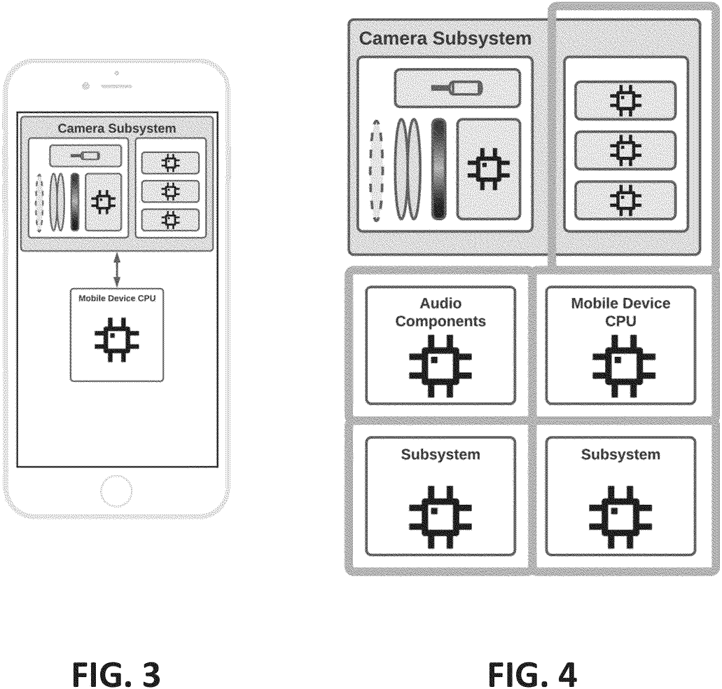

FIG. 3 illustrates the medical sensor subsystem integrated into a personal user device, such as the illustrated smartphone. In FIG. 3, the subsystem contained in the shaded box is the hardware that would be controlled (e.g., regulatorily controlled) as a “medical device,” while the rest is uncontrolled and part of each individual mobile device.

In some embodiments, in developing such a medical sensor subsystem, one or more of the following items should be specified and/or controlled as part of the medical device design. Personal user device manufacturers would be provided with this type of information such that the medical sensor subsystems could be integrated into the personal user devices. This information can include, for example:
PCB artwork - precise layout for all electrical components;
Mobile device CPU connections and/or protocols;
Cover lens - many mobile device cameras have an optically transparent cover lens. It may be part of the camera module, or it may be installed in the phone. If installed in the phone, it may need to meet a specification;
Physical mounting - some parts of mounting may need to be controlled to ensure stability and/or prevent occlusion;
Keepouts - may need to define minimum distances to other components in the phone, including antennas, high speed digital lines, or other radiation/noise sources;
EMI shielding - it is common in smartphone PCB designs to have various subsystems grouped onto specific areas of the PCB and have EMI shielding around them, due to the various RF energy sources on the phone (antennas for cell network, Wi-Fi, Bluetooth, NFC, etc.). It may be necessary to specify/control the design of a shielding system for the sensitive ICs and other components of the camera subsystem. This shielding can be part of the overall shielding solution of the PCB, as long as it satisfies specified performance parameters for the camera subsystem.

FIG. 4 is a diagram schematically illustrating additional components of the personal user device of FIG. 3 and shielding that can separate the camera subsystem from the other components, according to an embodiment. The various integrated circuits comprising the camera subsystem electronics are shielded, while the camera module itself is not.

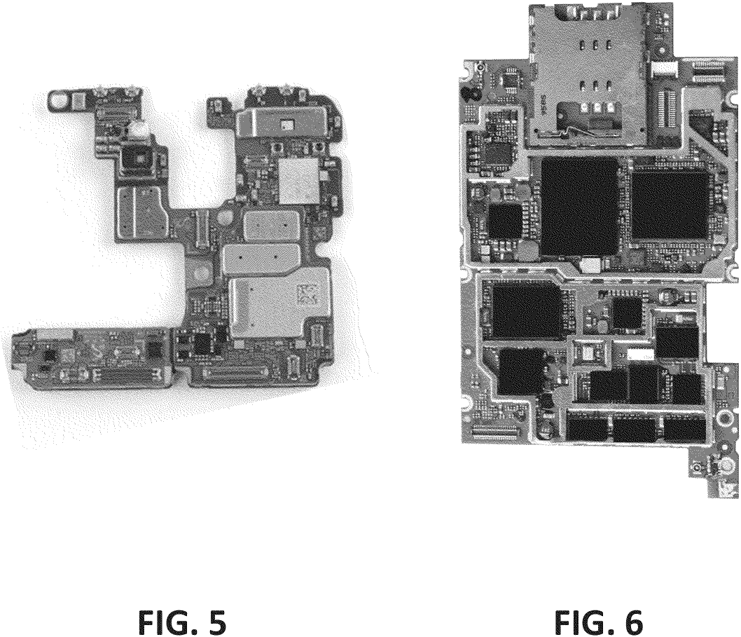

FIGS. 5 and 6 are examples of modern smartphone PCBs, showing subsystems arranged into shielded areas. In FIG. 5, metal caps are soldered onto a ground plane to comprise a shield. In FIG. 6, metal walls are soldered onto a ground plane, and then a metal plate/foil is installed on top of the PCB during assembly to complete the top of the shield.

In some embodiments, the software running on the personal user device CPU must not be able to change the software on the ISP or otherwise alter images before they reach the CPU. In other words, the images are passed from the medical sensor subsystem to the remote compute resource without alteration. Additionally, most medical devices are required to be calibrated at a regular interval. To achieve this, a simple printed card could be shipped to the user. The user could be instructed to run an app that would walk them through a process of taking images from different angles. From this, the calibration of the camera could be confirmed and/or updated. Such calibration may be necessary for compliance with the regulatory agencies. During manufacture, each unit may need to be subjected to a test suite to ensure proper function of the camera subsystem to the medical device specification in order to comply with regulatory requirements. In some embodiments, calibration of the camera may be required for use in medical-related applications, but not necessarily required for other uses (e.g., for taking pictures of friends, family, scenery, etc.). As such, in these embodiments, the user may only be instructed to perform a procedure for calibrating the camera if the user attempts to use the camera for a medical-related application and over a threshold amount of time has elapsed since the last time the camera was calibrated.

While an example of a medical sensor subsystem configured for imaging has been described above, other subsystems could similarly be produced.

For example, in some embodiments, a microphone or other audio recording subsystem can include one or more of the following components, among others: microphone hardware, preprocessing integrated circuits (if necessary), a printed circuit board (PCB) layout for noise and/or function, and or a case opening or porting.

In some embodiments, an inertial measurement subsystem can include one or more of the following components, among others: IMU hardware (e.g. accelerometers, magnetometers, and/or gyrometers or gyroscopes), preprocessing integrated circuits (if necessary), a printed circuit board (PCB) layout for noise and/or function, and or orientation and mounting structures.

In some embodiments, a pressure sensor subsystem can include one or more of the following components, among others: a pressure sensor, preprocessing integrated circuits (if necessary), a printed circuit board (PCB) layout for noise and/or function, and or a case opening or porting.

### Remote Image Quality Verification

The systems, methods, and devices described in this application can facilitate and enable the use of personal user devices, such as smartphones, tablets, laptops, personal computers, or other devices, for remote or at-home healthcare through the use of remote image quality verification. In some embodiments, images captured using personal user devices can be uploaded to a remote server, system, or other device (e.g., a device in the cloud) for image quality verification. The remote server can determine whether the image is of sufficient quality for use in a medical diagnostic or test. In some embodiments, the remote server can apply one or more image processing techniques and parameters to the image to produce a processed image of sufficient quality for use in the medical diagnostic or test.

As described above, in some embodiments, a personal user device can undergo a calibration procedure to ensure that an imaging system is performing within acceptable parameters. In some implementations, alternatively or additionally, image quality can be assessed remotely. For example, in some implementations, remote assessment may obviate any need for the user to perform a local calibration or verification procedure. However, even in the case where the user performs a calibration or verification procedure, it can be important to ensure that images received by the remote system are of sufficient quality (e.g., lighting, focus, and so forth) for interpreting test results, verifying that testing procedures were followed correctly, and so forth.

In some embodiments, performing image quality verification on a device that is remote or separate from the user’s personal device can provide several advantages over having each personal user device perform its own image quality verification. As one example, the remote device configured to perform image quality verification can be or can be configured to implement a regulatorily approved medical device or process (e.g., an FDA-approved device or process). Accordingly, in some embodiments, the remote image quality verification systems, methods, and devices described herein can provide an image verification platform that can ensure compliance with standards (e.g., regulatory standards or others) even when the images are captured by devices that may not themselves be compliant.

Additional advantages that can be provided in some embodiments can also include that, with the use of remote image quality verification, there need only be one version of the software that performs the image verification. In this way, the software can easily be kept up to date as it is only located on one device (or a group of related managed devices). By contrast, if image verification is performed on the personal user devices themselves, it would be exceedingly difficult to ensure that all devices are running the most up to date version of the software.

Additionally, providing remote image quality verification on a remote system or device can be more secure than providing image quality verification on each of a large number of different personal user devices that are owned and controlled by a wide number of different people. For example, some of the personal user devices could use operating systems that are outdated and include security flaws that can be exploited, or some of the personal user devices could be infected with spyware, malware, viruses, etc. In some cases, a user may tamper with or otherwise modify a personal user device.

Also, as noted above, there are a wide variety of different personal user devices from different manufacturers, each with different hardware and software configurations. Given the high level of diversity among all possible personal user devices, it can be hard to guarantee that software for performing image quality verification would run well or the same on different user devices. By providing image quality verification on a remote device, this problem can be reduced or eliminated.

Performing image quality verification on such a remote image verification platform may also save medical diagnostic test manufacturers the hassle of having to pursue regulatory approval by providing them with an already-approved off-the-shelf solution. For example, in some situations, the medical diagnostic test manufacturers may instead focus on receiving regulatory approval on techniques for interpreting test results based on images that are verified using the image verification platform. In some embodiments, test interpretation logic may be implemented on a personal user device or other medical device based on images that have been verified by the remote image verification platform.

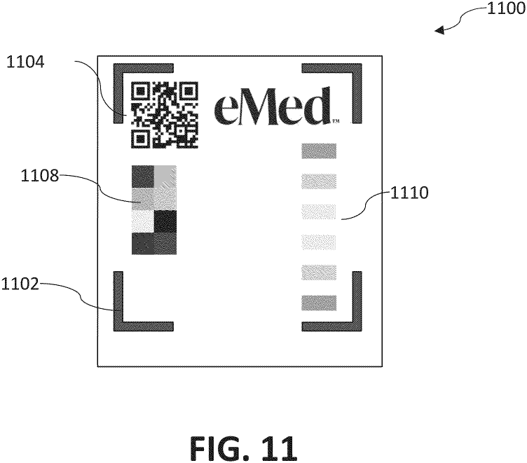

In some embodiments, the image verification techniques described herein may leverage an artifact within the image as part of the verification process. In some embodiments, the artifact can be a reference card within the image. An example reference card is described in more detail below, with reference to FIG. 11.

In some embodiments, the image verification techniques test the image quality of one or more images. If the images produced by the camera pass the test, then the quality of the camera and images may be deemed to meet the corresponding regulatory or other requirements. If the images fail, then the quality of the camera and images may be deemed to not meet the corresponding regulatory or other requirements. In some embodiments, upon failure, the user may be notified accordingly. For example, the user may be requested to provide new images taken with a different device or with better conditions (e.g., lighting).

In some embodiments, the image verification platform may be able to evaluate other aspects of each personal user device to determine whether or not the user device is sufficiently capable of capturing images for use in medical diagnostics that meet the necessary requirements. For example, the user device could indicate its make and model (e.g., MAC address, device ID, etc.) and/or other specifications of the user device, and the image verification platform could determine whether these specifications are sufficient. If the specifications are deemed to be sufficient, then the image verification platform may select a calibration profile associated with the specifications of the user device that is to be used as a baseline calibration profile for calibrating images captured by the user device. In some embodiments, the parameters of the profile can be modified throughout the process. In some embodiments, at least one score could be generated by the image verification platform for each personal user device that is representative of a level of sufficiency of the imaging capabilities of the user device (e.g., results threshold detection score, sharpness score, etc.) can be determined.

At a high level, remote image quality verification can include prompting a user to take an image, for example, of a reference card, using a camera of his or her personal user device. The image of the reference card can be uploaded by the personal user device to a server on the cloud. The server can be configured to evaluate the image to determine whether the image is already of sufficient quality (for being used in medical diagnostics under FDA or other regulatory requirements), or the server can process the image using designated processing techniques and parameters to produce a processed image of sufficient quality. Finally, the server can respond to the user device with an indication of the determination result.

In some embodiments, remote image quality verification can be performed prior to image analysis from which a test result or medical diagnosis can be determined. For example, a user can be instructed to capture an image of a test strip using a camera of his or her personal device. The image of the test strip can be uploaded by the personal user device to a server on the cloud. The server can be configured to evaluate the image to determine whether the image is already of sufficient quality (for being used in medical diagnostics under FDA or other regulatory requirements) or the image can be processed using designated processing techniques and parameters to produce a processed image of sufficient quality. Finally, the server can respond to the user device with an indication of the determination result. If the quality is sufficient, computer vision or other techniques can be applied to the image to determine a test result or diagnosis. If the quality is insufficient, the user can be connected with a live proctor for results interpretation or other instruction.

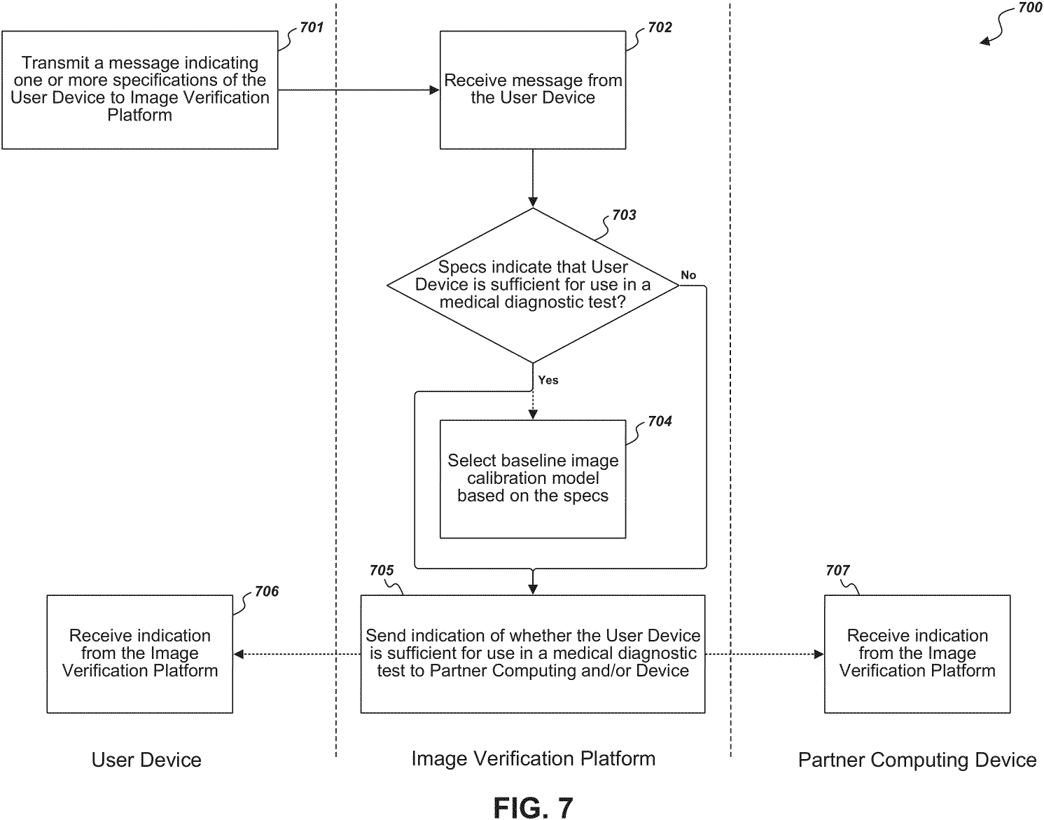

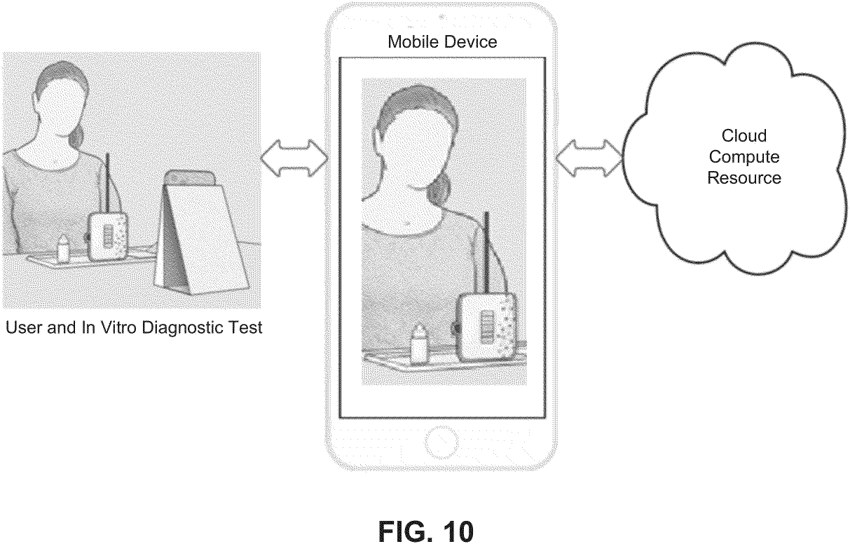

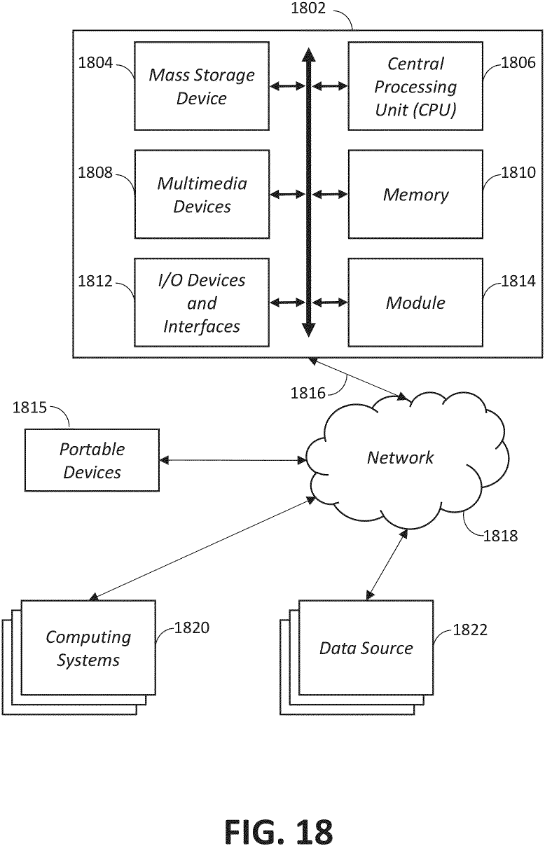

FIG. 7 illustrates an example process 700 that can, in some embodiments, be implemented for remote image verification. In the illustrated embodiment, certain steps are performed on a user device, certain steps are performed on an image verification platform, and certain steps are performed on a partner computing device. The user device can be, for example, a personal user device including a camera, such as a smartphone, tablet, laptop, personal computer, or other device (e.g., the smartphone of FIG. 10 or the portable device 1815 in FIG. 18). The image verification platform can be a hardware and or software platform implemented on a device separate from, and in some instances, remotely located from the user device. For example, the image verification platform can be a remote server, such as a cloud-based server (e.g., the cloud compute resource of FIG. 10 or the system 1802 of FIG. 18). The partner computing device can be another remotely located device (e.g., computing system 1830 of FIG. 18). In some embodiments, the process 700 can be performed prior to beginning a remote medical diagnostic test that will make use of an image captured with the camera of the user device. The process 700 can be configured to determine if the camera and/or the user device are sufficient for use during the medical diagnostic test.

In the illustrated embodiment of FIG. 7, the process begins at block 701, at which the user device transmits a message indicating one or more specifications of the user device to the image verification platform. The specifications can include information about the user device, such as the make, model, hardware configuration and components, software version, MAC address, device ID, etc. At block 702, these specifications are received by the image verification platform and the process moves to decision state 703. At decision point 703 the image verification platform determines whether the received specifications of the user device indicate that the user device is sufficient for use in a medical diagnostic test. In some embodiments, the determination at decision 703 is based on, for example, comparing the specifications received at block 702 to a database of acceptable user devices.

If, at decision state 703, the image verification platform determines the user device is sufficient, in some embodiments, the image verification platform, at block 704, optionally, selects a baseline image calibration model based on the specifications. The image calibration model can provide image parameters (e.g., brightness, contrast, white balance, saturation, etc.) for calibrating or adjusting an image captured by the user device.

At block 705, the image verification platform sends an indication of whether the user device is sufficient for use in the medical diagnostic test to the user device and the partner computing device. The user device receives the indication at block 706 and the partner computing device receives the indication at block 707. The indication can be positive, enabling use of the user device for the medical diagnostic test, or negative. In the event of a negative determination, the user device may, in some embodiments, not be useable for the medical diagnostic test.

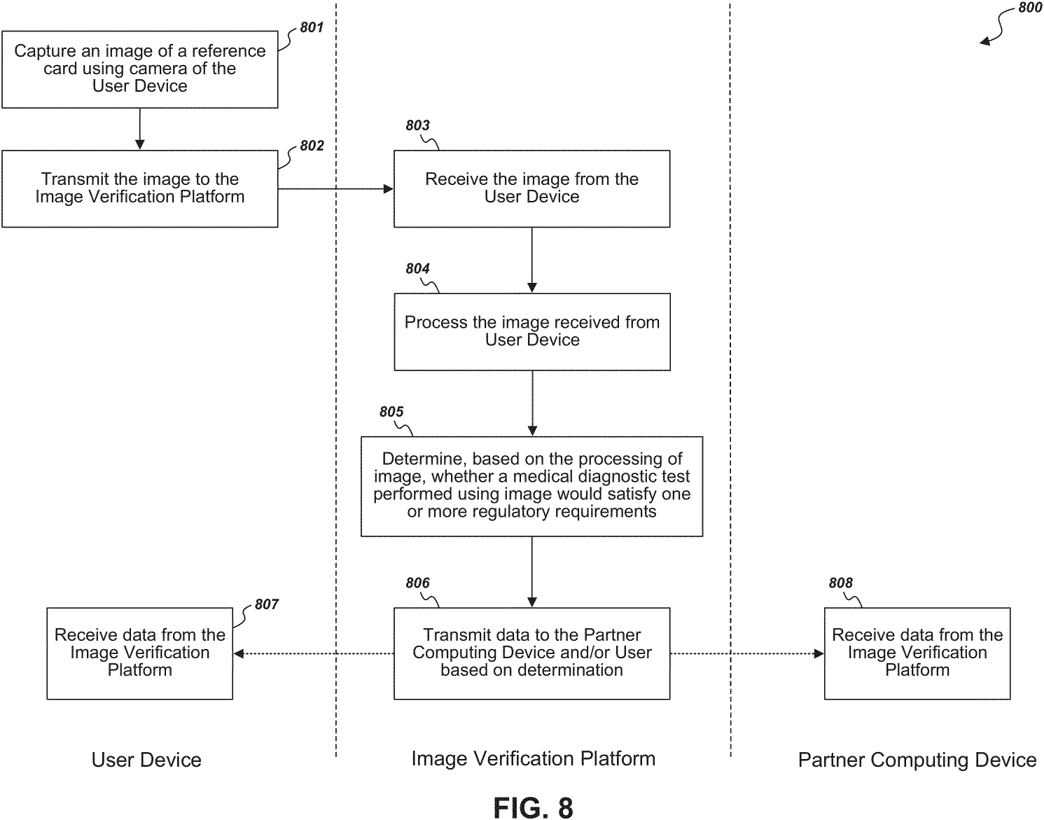

FIG. 8 illustrates an example process 800 that can, in some embodiments, be implemented for remote image verification. As before, in the illustrated embodiment, certain steps are performed on the user device, certain steps are performed on the image verification platform, and certain steps are performed on the partner computing device.

In the illustrated embodiment, the process 800 begins at block 801 at which the user device captures an image of a reference card. An example reference card is shown below in FIG. 11. Although this example relates to an image of a reference card, other images can also be used, including images of other testing materials, images of the user, etc. At block 802, the user device transmits the image to the image verification platform, which receives it at block 803.

At block 804, the image verification platform processes the image. In some embodiments, the processing operations of block 804 may include applying an image calibration model to the image to perform color correction, etc. In some of these embodiments, the image calibration model used at block 804 may correspond to that which is selected at block 704 in process 700.

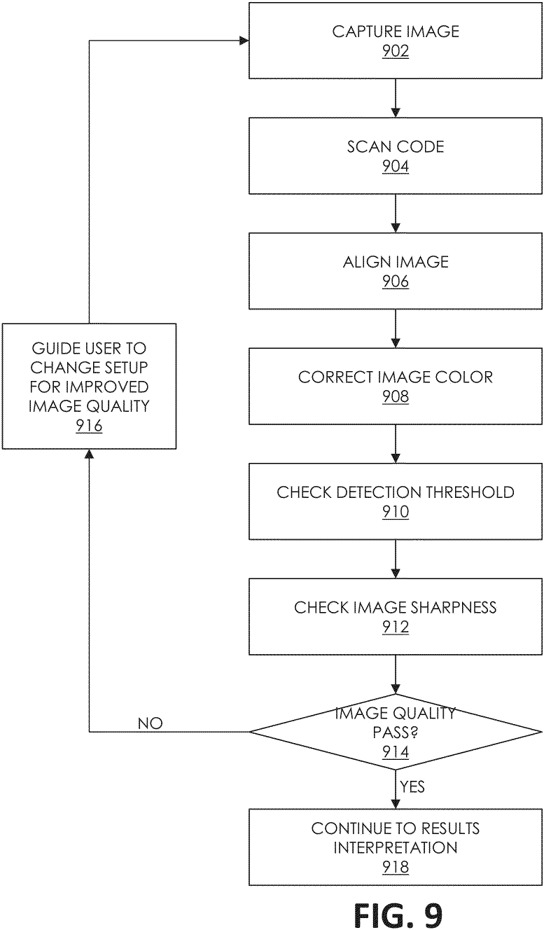

At block 805, the image verification platform determines, based on the processing of the image, whether a medical diagnostic test performed using the image would satisfy one or more regulatory or other requirements. In some embodiments, the operations of block 805 in process 800 may include evaluating the image as processed using the image calibration model against a set of criteria. Examples of the calibration and/or correction operations that can be performed at step 805 are shown in FIG. 9. At block 806, the image verification platform transmits data to the user device and the partner computing device, where it is received at blocks 807 and 808, respectively.

In some embodiments, in response to a determination at step 805 that a medical diagnostic test performed using the image would indeed satisfy one or more regulatory requirements, a set of one or more operations for determining a test result in connection with said medical diagnostic test may be performed based at least in part on the image. In some of these embodiments, the aforementioned set of one or more operations may include analyzing the processed image using one or more image processing techniques (e.g., computer vision), for example, to determine a test result. In some of these embodiments, the aforementioned set of one or more operations may be performed by the image verification platform following step 805. In some such embodiments, the data that is transmitted by the image verification platform at step 806 may include the determined test result. In some of these embodiments, the aforementioned set of one or more operations may be performed by the partner computing device following step 805. In some such embodiments, the data that is received by the partner computing device at step 808 may include one or both of the image obtained by the image verification platform at step 803 and the processed image obtained or produced by the image verification platform at step 804. In some embodiments, the data that is transmitted by the Image Verification Platform at step 806 may include one or more of the image, the processed image, an indication of the result of the determination made at step 805, image calibration parameters, and a test result as determined in connection with said medical diagnostic test based at least in part on the image

In some embodiments, the process 700 is performed prior to the process 800. In some embodiments, the operations of blocks 801-803 in process 800 are performed responsive to the operations of one or more of blocks 705–707 in process 700. In some embodiments, the partner computing device may be entirely optional and/or not involved in the operations of one or both of processes 700 and 800.

In some embodiments, in response to a determination at block 805 that a medical diagnostic test performed using the image would indeed satisfy one or more regulatory requirements, the image verification platform may perform one or more operations (following block 805) to provide the image and/or processed image to a proctor (not depicted) for manual and/or human-based test results interpretation. In some embodiments, the image verification platform may additionally perform one or more operations (following step 805) to connect the user device with a proctor.

As mentioned above, FIG. 9 is a flowchart that illustrates various processing, correction, and/or calibration processes that can be performed on a captured image. In some embodiments, the steps illustrated in FIG. 9 can be used for preprocessing an image before determining results. However, the steps of FIG. 9 can additionally or alternatively be used at other stages in the testing process, such as at the beginning of the process to determine if lighting conditions, camera quality, etc., are sufficient for carrying out the testing session. At 902, the method can begin with an image of a reference card and a results strip being captured by a user device (e.g., a smartphone). At 904, a computing system can recognize an identifier, such as a QR code, bar code, or other distinct code, on the reference card. In some embodiments, the code or unique identifier can be recognized using a computer vision algorithm. The QR code or other code can be a unique identifier for the test and can be different for each instance of a test that is manufactured. The identifier can be used to track the test throughout the testing process and to mitigate some types of fraud. In some embodiments, the code can include manufacturing information such as the lot number, which may be used to track manufacturing defects. In some embodiments, the identifier can include an expiration date that can be validated before testing. In some embodiments, the identifier can include a test type, version, etc., that can be looked up in a database to determine test strip interpretation information such as, for example, where various reference card features and/or test strip lines should appear, to determine a testing procedure, and so forth. In some embodiments, instead of or in addition to embedding some types of information in the identifier directly, the identifier can include information (e.g., a unique ID) that can be used to query an external source, such as an external database, to retrieve information such as the lot number, expiration date, test type, and so forth. In some embodiments, the identifier may not be unique. For example, the identifier may be used to identify a type of test, a manufacturing date range, a version of a test, and so forth, but may not be used to identify specific instances of a test.

At 906, a computing system can be configured to align the image using one or more fiducials on the reference card. In some embodiments, the fiducials may be distinct features, while in other embodiments, the fiducials may be included in other features of the reference card, for example within the QR code. Aligning the image can include, for example, deskewing, removing keystoning, and so forth. At 908, the system can perform color corrections to the image, for example using known reference colors printed onto the reference card. At 910, the system may check to ensure that the user’s device is capable of detecting a range of color shades (e.g., from light pink to dark pink) that are printed on the test card (for example, to ensure that the user’s device can be used to detect a faint strip of color on a test strip). For example, a reference card can include a detection threshold calibration region having multiple color samples ranging from very light to relatively dark. At 912, the system may check the sharpness of the image using a computer vision algorithm. After checking one or more of the above indicators of image quality, the system can decide if the image is of sufficient quality to be used in assisting with test result interpretation at 914. If the image is not of sufficient quality, the system can, at 916, prompt the user to take another photo with better lighting, less motion, no obstructions, etc. The new image can be processed through the same method to check for image quality. If the image passes the quality check, the image may continue on and be used in a results interpretation step 918 where the test strip is compared to colors and/or shades printed on the reference card or to a reference that can be used to determine if a result is positive or negative.

FIG. 10 illustrates an example environment in which the processes 700 and 800 can be implemented. As shown, a user utilizes a personal user device, such as the illustrated smartphone, to take a medical diagnostic test (left pane). In the illustrated example, the smartphone is positioned in a stand which orients the smartphone relative to the user and the other components of a test kit. For example, the smartphone can be oriented such that the user can see the display of the smartphone (on which can be displayed testing instructions, communications with a proctor, etc.) and a camera of the smartphone (e.g., a forward-facing camera) has a view of the user and the test kit materials (such that a remote proctor or system can monitor the testing procedure).

As shown schematically in the center pane, the smartphone can capture an image during the medical test. As illustrated in the right pane, the smartphone can be in communication with remote compute resource, such as the illustrated cloud compute resource. Communication between the smartphone and the remote compute resource can occur over one or more computer networks, including over the internet. The remote compute resource can be configured to implement the features provided by the image verification platform in FIGS. 7 and 8.

An example reference card and details of some embodiments are shown in FIG. 11. In some embodiments, a test strip or other test result indicator can be positioned in front of the reference card (e.g., between the reference card and the user’s personal device (e.g., smartphone). The reference card can be a printed piece of cardstock, plastic, etc., with various elements and may be designed to include or exclude elements depending on the test type or other factors. In some embodiments, the card can serve as a background when capturing an image of a test strip or similar test kit component, such that all results images can be standardized. In some implementations, the card can provide a unique code 1104 (e.g., a QR code) that can be quickly and reliably identified and scanned using a computer vision process. In some test methods, the code can be scanned before a user takes a test and after the user takes the test, for example to ensure test continuity and security. The code can be referenced to a database containing information about the test such as lot number, expiration date, etc.

Various features of the reference card 1100 that can aid in obtaining an optimal camera image, from which results can be interpreted (e.g., by a trained proctor or a computer vision algorithm, AI model, and/or the like), can be included. For example, graduated color strips 1110 (which may be, for example, of a constant hue and varying saturation and/or brightness) can be printed on the card, and an image that includes the strips 1110 can be provided to a computer vision algorithm. If the algorithm can identify all shades of stripes, lightest through darkest, that image can be considered to have sufficient quality for accurately detecting the result stripes. Similarly, color references 1108 may be included. The color references may be blocks or other printed areas that include known colors on which color calibration (e.g., white balance, contrast enhancement, etc.) may be based. In some embodiments, a system can be configured to extract graduated color stripes and/or color references from an image. In some embodiments, the system can recommend changes to lighting conditions, distance, etc., to improve image quality. In some embodiments, the system can adjust the color of the images using the extracted color references and known color references. This calibration step may assist results in performing accurate test result interpretation.

Various fiducials 1102 may be included on the card to aid in position calibration of the image and to provide an image post-processing algorithm with a basis upon which distortion correction may be performed (for example, removing a skew or keystone effect). One or more of the features described above may be used as part of an image pre-check before the user completes the testing process. The pre-check may use the features to determine whether adequate lighting is present, whether angle adjustments of the user device are needed, or whether other image quality adjustments are needed. If one or more of the pre-check items needs adjusting ahead of the test session, the image check algorithm or the test system may indicate to the user to make such adjustments (e.g., “please turn on a brighter light” or “please rotate the kit to face the nearest window”).

### Self-Guided Testing

The systems and methods described above can play an important role in enabling remote medical or diagnostic testing. Users of remote or at-home medical testing may often be able to complete a test without needing assistance from a proctor. However, some form of review by a proctor and/or remote compute resource may be needed to ensure that a test is valid. For example, a proctor or remote compute resource may need to make sure that swabs were collected properly or that the right person took the test (for example, in the case of a remotely administered screening for illicit drugs). While review by a proctor may be needed for some parts of some tests, requiring a proctor to be present for the entire duration of a test may result in delays, added expense, and user frustration. For example, a user may have to wait for a proctor to become available, a testing provider may have to hire additional proctors, and so forth.

During administration of some tests, there may be no live proctor during a testing session. For example, a user may complete tests in a self-guided manner. In some embodiments, to facilitate the user completing the tests in a self-guided manner, the user may be instructed using written instructions, voice-based instructions, augmented reality (AR)-based guidance, video-based guidance, or the like. The instructions can be provided to the user on a user device, such as a smartphone, a tablet, a laptop, a personal computer, or the like, on which the user accesses a remote healthcare platform (e.g., through an application or website) that facilitates the testing session. In some embodiments, during a testing session, video of the testing session may be captured for later review, for example, using one or more cameras on the user’s user device. During testing, the user may be directed to place testing materials and/or the user’s head or other body part within the field of view of the camera of the user’s device. In some embodiments, augmented reality and/or computer vision may be used to help the user capture video that is suitable for proctor review. In some embodiments, a pre-test process can include verifying that the user’s device is suitable for taking a remote medical or diagnostic test, for example as described above, for example by verifying one or more properties of the device, verifying a sample or calibration image, and so forth.

If the test involved in the testing session requires some level of review or oversight, in some embodiments, a proctor may review an abbreviated version of the captured video. The proctor can be associated with the remote healthcare platform and can access the remote healthcare platform with a proctor device that itself can be a smartphone, tablet, laptop, personal computer, or the like. The proctor can access the recorded video of the testing session and review it to ensure that the user performed the self-administered test correctly, verify the user’s identity, or the like.

In some embodiments, video of a testing session may be played back (e.g., by or for the proctor) at a plurality of frame rates or a plurality of video speeds. For example, in some embodiments, a first phase of the testing session may be played back at a first frame rate or speed and a second phase may be played back at a second frame rate or speed. In some instances, portions of the test that require closer review (e.g., critical or important testing steps, identification steps, and so forth) can be played back and reviewed at a lower frame rate or slower speed, while portions of the test that require less review (e.g., less critical or important steps, such as waiting steps) can be played back and reviewed at a higher frame rate or faster speed. In some embodiments, for example, the first phase may correspond to test setup and sample collection and may be played back at a relatively low frame rate (for example, at normal speed). This can allow the proctor to review these portions of the test very carefully and accurately to ensure adequate compliance and accuracy. In some embodiments, the second phase may, for example, correspond to a waiting period and may be played back a higher frame rate, such as twice the normal speed or four times the normal speed, in order to minimize the time a proctor spends reviewing video that is unlikely to contain events that are consequential to the outcome of the testing session. In some embodiments, a proctor may review the entire testing session (e.g., through a combination of video playback at different speeds). In some embodiments, a proctor may review only some parts of a testing session (e.g., some portions of the recorded testing session may be omitted for proctor review).

Accordingly, in some embodiments, the proctor may review an abbreviated video of the testing session after one or more testing phases (e.g., a video whose length is less than the total real-time length of the testing session), allowing for an increase in proctor efficiency. During review, the proctor may determine that the testing session should continue (e.g., when steps of the test have been performed correctly and have been able to be verified) or that a problem has occurred with the test (e.g., when one or more steps have been performed incorrectly or are otherwise unable to be verified) and a new test needs to be administered or some other intervention made.

In some embodiments, proctor review of the testing session is performed in real time or substantially real time (for example, within a few minutes of the testing session), such that proctor feedback to the user may allow a defective testing session to be corrected and continued by, for example, repeating one or more steps. For example, the proctor can intervene with the user for example by asking that one or more steps be repeated in a verifiable manner. In some embodiments, however, the testing session may be terminated early because, for example, an error occurred during the testing procedure that is not correctable, requiring the testing session to be aborted and restarted. For example, the proctor, during review (substantially in real time or later) may indicate that the user has or has not complied with instructions by, for example, selecting a button on an interface. In some embodiments, the proctor may not review the video of the testing session until after the session is completed.

In some embodiments, during testing, the user may encounter difficulty while taking a test. For example, the user may be unsure of how to comply with and perform one or more steps of the tests according to the provided instructions. In such cases, in some embodiments, the user may be able to request assistance. For example, in some embodiments, the user may be able to access more detailed instructions or may be provided with instructions in a different format such as, for example, audio, text, video, augmented reality, or the like. In some embodiments, the user may be able to request assistance from a live proctor. In some embodiments, testing steps that have a higher likelihood of error may be accompanied with animated and/or video demonstrations. In some embodiments, the user can request to be connected with a proctor for receiving additional assistance. The proctor can be a live proctor or an artificial intelligence-based proctor. For example, a user may first be provided with an AI-based proctor and may subsequently be escalated to a live proctor.

In some embodiments, to further facilitate review of testing sessions, proctors may be shown video alongside overlays, voiceovers, animations, or example videos that may help the proctor to identify problems with the testing session. In some embodiments, proctors may be able to watch video at higher than normal speeds (e.g., at 1.25x, 1.5x, 2x, 2.5x, 3x, 3.5x, 4x, or any number between these numbers, or more or less). In some embodiments, proctors may be able to scrub through video and/or rewatch sections of a testing session. For example, in some embodiments, individual steps or sets of steps may be identified within the video and a proctor may be able to easily jump from section to section. In some embodiments, markers may be added, for example, when the testing user advances from one step to the next step.

In some embodiments, a user who has completed a test may be directed to participate in a test result interpretation procedure. In some embodiments, a proctor may review the results with the user. In some embodiments, the user may review the results alone.

### Self-Guided Test Result Interpretation

As mentioned briefly above and as will now be explained in more detail below with reference to the example embodiments provided in the figures, users can engage in self-guided results interpretation, such as health testing or diagnostic results interpretation.

As discussed above, a user or patient may administer a medical exam, health or diagnostic test, or the like, with the use of a user device (such as a cellphone, smartphone, tablet, laptop, personal digital assistant (PDA), or the like). The administration of the diagnostic test may produce results that may need interpretation. The user may be prompted, for example, on the user device, to capture information of the medical diagnostic test that relates to the results of the medical diagnostic test and to indicate their interpretation of the results of the medical diagnostic test. Such information can include, for example, pictures, videos, audio recordings, or the like, that relate to the diagnostic test. For example, the user may be asked to take a photo of a test strip, test card, and/or the like. The user may be instructed to take such a photo by placing the test strip, test card, etc., in front of a reference card as described above. The user may indicate their interpretation of the results and can be provided with guidance that may include explanations, references to example results, diagrams, step-bystep instructions, etc. A system, such as a remote healthcare or proctoring platform, can be configured to receive the information provided by the user on the user device. The system can further be configured to present such information to, for example, a proctor, through the use of a proctor device (such as a cellphone, smartphone, tablet, laptop, PDA, or the like). In some instances, the proctor may be prompted, for example on the proctor device, to indicate or confirm their interpretation of the result of the medical diagnostic test shown in the captured information. Once the proctor’s indication has been made, the validity of the medical diagnostic test may be assessed accordingly. This may involve additional communications with the user that can lead to further interpreting the results.

The handling of results and result interpretation can be time intensive for proctors. Accordingly, it may be beneficial to provide self-guided test result interpretation, wherein the user is guided through a self-interpretation of the test results, for example, to decrease or eliminate proctor time involved in the process. This can simplify the process on both the user’s end and the proctor’s end, providing an improved experience for both. Additionally, this can allow the user to move at their own pace during a testing session. Further, personal and health information of the user may be protected because, in some embodiments, a proctor may later review the user’s self-interpretation without needing to know the user’s identity. For example, in some cases, the proctor may review the user’s self-interpretation anonymously. By providing the user with a guided opportunity to interpret the results of the medical diagnostic test, the user may indicate their interpretation of the results and a proctor may confirm or deny the results to issue or invalidate the results in an efficient manner.

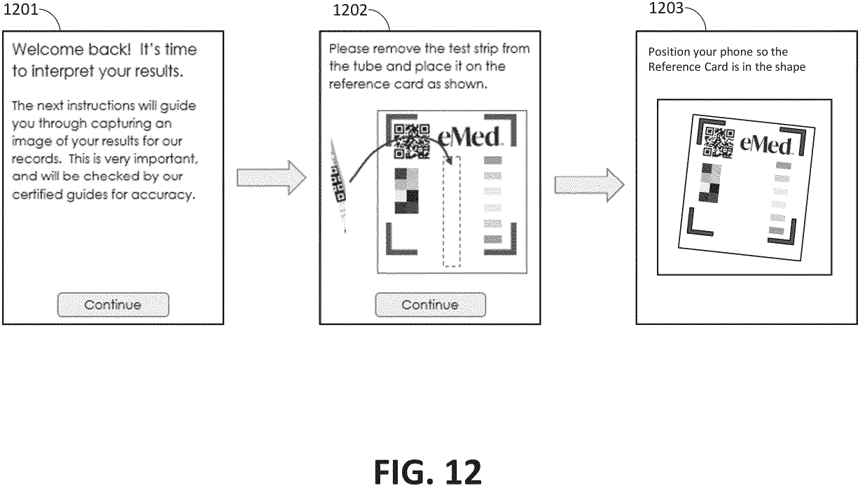

FIGS. 12-14 illustrate an example embodiment of a user interface that guides a user through making a self-interpretation of their test results and submitting the results to a healthcare or proctoring platform where the self-interpretation can be reviewed by a proctor to either confirm or deny the user’s self-interpretation. The user may first be prompted, on a user device, that it is time to interpret the results of their medical diagnostic test, as illustrated in screen 1201. The user may be provided with instructions to guide them through the process leading to result interpretation. The user may choose to continue with the process by clicking “continue.” The user may then be prompted to begin preparation to interpret the medical diagnostic test results at screen 1202. Such preparation may include the removal of a test strip from a medical diagnostic test and the placement of the test strip on a reference card provided with the medical diagnostic test. The user may select continue to proceed. The user may then be prompted to position the user device to allow the user device to capture an image of the reference card at screen 1203. The user may be prompted to place the reference card within the visual field or within a part of the visual field of a camera of the user device that may further place the reference card within a shape provided on a screen of the user device. The user device may then capture an image of the medical diagnostic test strip. In some embodiments, the user may manually trigger capturing of the image, for example by tapping a button. In some implementations, a system can be configured to automatically recognize when the reference card is positioned correctly and can automatically capture the image. For example, the system can be configured to recognize fiducials or other features of the reference card as discussed above with reference to FIG. 11.

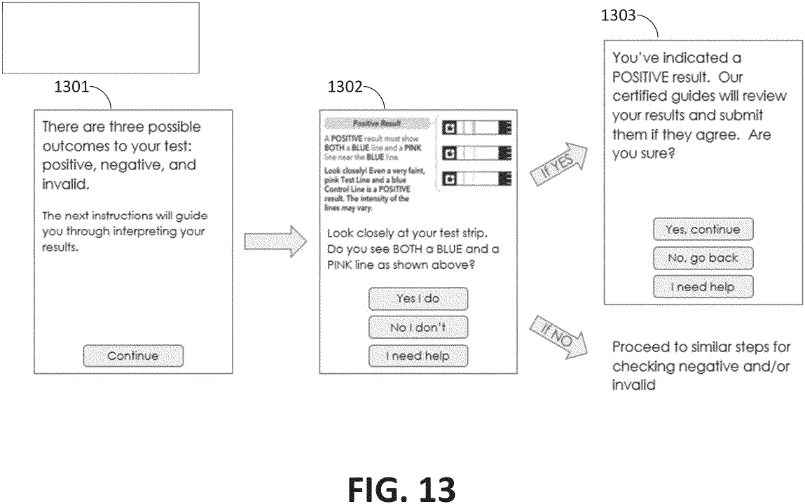

As shown in FIG. 13, the user may be prompted that there are three possible outcomes to the medical diagnostic test at screen 1301. The three possible outcomes may include positive, negative, and invalid. In some implementations, other outcomes may be possible, such as indeterminate. The user may continue with the self-guided result interpretation by clicking continue. At screen 1302, the user may be provided with examples of what may be considered a positive or negative result. Such examples may include explanations of what the user may see on their own test strip or example diagrams of what the user may see on their own test strip. The user may be prompted to indicate how they interpret their test result. For example, the user may be asked whether they see both a blue and a pink line. In this example, the user will then select that they do see both a pink and blue line or that they do not see both a pink and blue line. The user may also select that they need help. It will be appreciated that is example is merely illustrative and is not limiting. For example, some tests may not use color to differentiate between a control line and a test result line. Some tests may not have a control line and may instead rely on some other mechanism to indicate the validity of the test result (e.g., color, size, shape, etc.).

In some embodiments, if the user indicated that they interpreted a positive result of the medical diagnostic test, the user may be prompted at screen 1303 that a proctor or certified guide will review the results and submit the results if the proctor agrees. The user may then select to continue with the result interpretation process, go back, or select that they need help. In some embodiments, if the user indicated a negative result because they did not see both a blue and a pink line, the user may be prompted with a screen similar to screen 1303 that they have indicated a negative or inconclusive result and that a proctor or certified guide will review the results.

In some embodiments, results interpretation may proceed in a manner that is different from that depicted in FIG. 13. For example, instead of being asked at 1302 if the user sees both a pink test line and a blue control line, the user may be asked if they see a blue control line. The absence of a blue control line can indicate an invalid result. If the user indicates that they see a blue control line, a subsequent step can include asking the user if they see a pink test line, which can indicate a positive (e.g., test line present) or negative (e.g., test line absent) result.

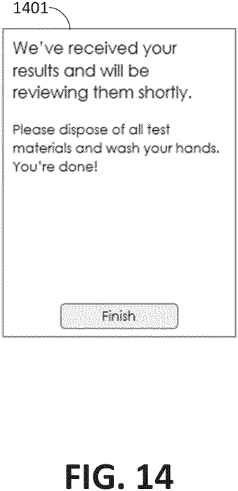

As shown in FIG. 14, the user may be provided with a confirmation screen 1401 that their results have been received and will be reviewed shortly. The user may be prompted to dispose of all test materials, to wash their hands, and/or conduct other post-test activities. The user may further be prompted that their results are processing and notified when the processing is complete. For example, the notification may include the sound of a chime, ping, bell, etc. In some embodiments, the user may receive a push notification, text message alert, email, or other form of notification indicating that processing is complete.

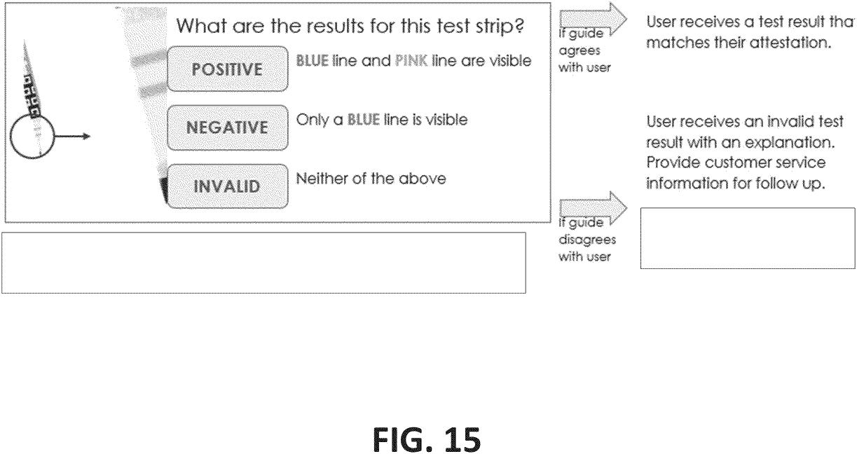

FIG. 15 illustrates an example embodiment of a proctor interface configured to allow a proctor to indicate their interpretation of the result of the user’s medical diagnostic test. The proctor may review the user’s attested results anonymously in the background. For example, the proctor may review the user’s attested results after the user has provided them, such that direct (e.g., real-time or live communication) communication between the user and the proctor is not necessary. In some embodiments, the proctor may review the user’s attested test results without knowing the identity of the user, making the testing process generally anonymous such that the user’s personal identifying information is unknown to the proctor. Such anonymity can allow the user’s identity to remain anonymous while the proctor reviews the medical diagnostic test results. The proctor may be prompted in asking for an indication of whether the proctor sees a blue and pink line, only a blue line, or neither a blue and pink line or a blue line. That is, the proctor may see a control line and test result line (e.g., indicating a positive result), a control line and no test line (e.g., indicating a negative result), neither a control line nor a test line (e.g., indicating an invalid test), or a test line and no control line (e.g., indicating an invalid test). In some embodiments, the proctor may be unaware of how the user interpreted the results.

In some embodiments, if the proctor interpreted the results of the medical diagnostic test to conclude the same outcome as the user’s interpretation of the results, then the user may receive a test result (e.g., an official or final test result) that matches their self-attestation. In some embodiments, if the proctor interpreted the results of the medical diagnostic test to conclude a different outcome than the user’s interpretation of the results, then the user may receive an invalid test result with an explanation. In this embodiment, the user may be provided with contact information for follow up, for example, with the proctor or a healthcare professional.

In some embodiments, if the user attests a positive result of the medical diagnostic test and the proctor attests a negative result, a positive result may be reported unless the medical diagnostic test is clearly invalid. In some embodiments, upon a disagreement between the user and the proctor, a contact representative may connect with the user to guide the user to the proctor’s interpretation of the test results. In some instances, the contact representative may explain how the proctor interpreted the user’s test results to provide the user an opportunity to change their interpretation of the test results. In some instances, a system may be configured to escalate review of the test results to a results interpretation algorithm. The results interpretation algorithm may use computer vision and/or machine learning techniques to interpret the test results in a computerized manner. In some embodiments, the results interpretation algorithm may interpret the test results whether the proctor and the user conclude the same or different interpretations of the test results. In some embodiments, the results interpretation algorithm may resolve a disagreement in interpretation of the test results between the proctor and the user. In some embodiments, if the results interpretation algorithm concludes a different result than the proctor concluded, thereby disagreeing with the proctor, then the test results may be analyzed or reviewed by a second proctor for their interpretation of the results. In some embodiments, test identifying information (e.g., a QR code, barcode, etc.) may be used to verify the same test kit component has been used throughout the duration of the administration of the test.

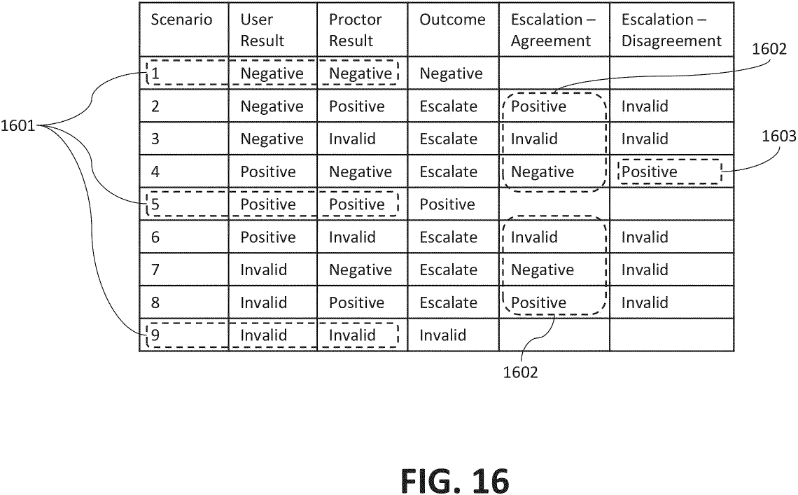

FIG. 16 illustrates an example embodiment of the results interpretation logic. In some embodiments, the user will make a self-guided results attestation (“User Result”) for their medical diagnostic test, and a proctor will asynchronously make a results attestation (“Proctor Result”) for the same medical diagnostic test based on the information captured by the user’s device. For example, the proctor may review the medical diagnostic test results at a different time than the user attested its interpretation of the results. In some embodiments, if the user and the proctor attest the same result, then a report may contain the agreed upon result. For example, as indicated in boxes 1601, the user result and proctor result can both be positive, both be negative, or both be invalid. In these cases, there is no disagreement to resolve, and an outcome can be determined without further review or escalation. In some embodiments, the results interpretation algorithm may interpret the test results in the background. In some embodiments, if the user and the proctor attest different results, then the user may connect to a contact representative who may attempt to guide the user to interpret their results to a more accurate indication. In some instances, a system configured to escalate review of the medical diagnostic test results to a results interpretation algorithm that uses computer vision or machine learning techniques will interpret the results of the user’s medical diagnostic test. If the user is convinced to accept the proctor’s attestation of the results and the results interpretation algorithm interprets the results to conclude the same outcome as the proctor, then an escalation agreement may be noted as the agreed upon result, as indicated in the boxes 1602. If the user is not convinced to accept the proctor’s attestation of the results and the results interpretation algorithm interprets the medical diagnostic test result to attest the same or different outcome than the proctor concluded, then an escalation disagreement may be noted as invalid. In some embodiments, if a user indicates a positive test result and the proctor indicates a negative result, and no subsequent agreement can be reached, a report can be generated indicated a positive result, as indicated in box 1603, although some embodiments may not indicate a positive result when there is an outstanding disagreement between the user and the proctor. For example, a user may insist that they see two lines on a test strip even though the proctor does not see both lines. A test line may, for example, be too faint to be detected in an image provided by the user. In some embodiments, the user may be prompted to retake the medical diagnostic test that may provide the user with the option to order or otherwise be issued a new medical diagnostic test kit. In some embodiments, the escalation disagreement may be noted as positive if the user is convinced they see two lines and the results interpretation algorithm interprets the results to conclude a positive outcome.

In an alternative implementation, the “Proctor Result” column can indicate a final determination on the part of the testing provider. For example, the Proctor Result column can indicate the test result after a test is checked using a results interpretation algorithm, after a second proctor verifies a result, and so forth. As above, when the user result and proctor result agree, the agreed-upon result can be included in a generated report. If there is disagreement, an escalation can include prompting the user to re-check their test result. If the user subsequently indicates agreement, the agreed-upon result can be included in a generated report. If the user and testing platform do not reach an agreement, the result can be invalid, except optionally in the case where a user indicates a positive result and the platform attests to a negative result.

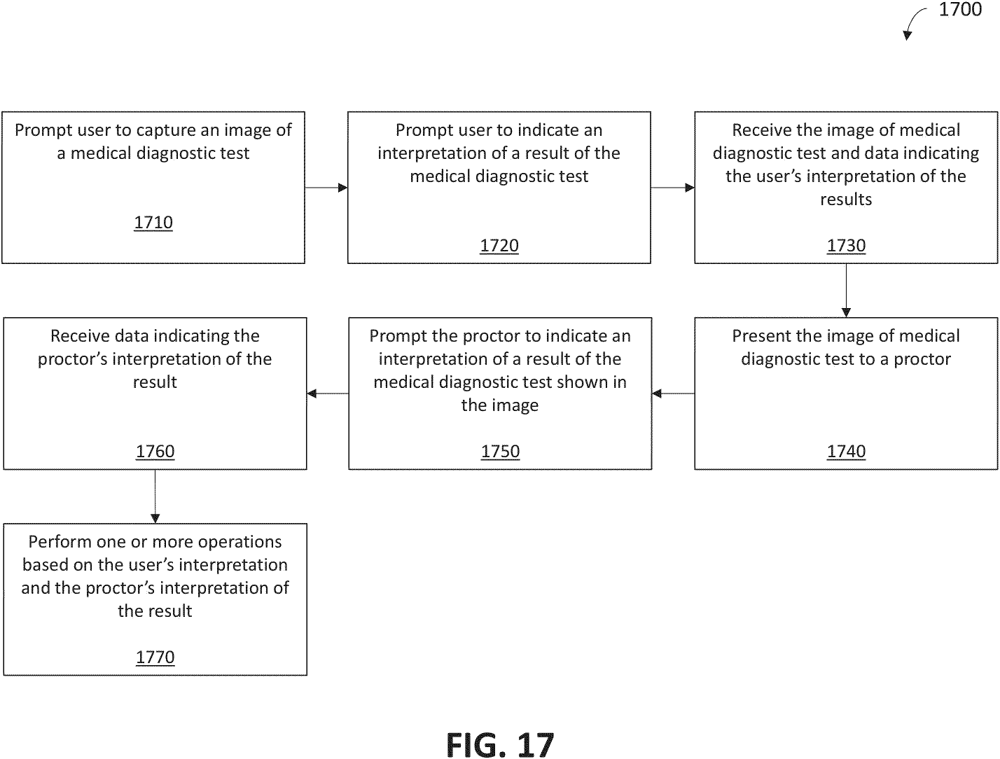

FIG. 17 is a block diagram illustrating an example self-guided results interpretation protocol or method. The method 1700 can be configured to facilitate self-guided interpretation of medical diagnostic test results. The method 1700 can be implemented on a user device or proctor device. The user device can be configured to guide a user through a self-guided interpretation of results, such as results of a medical diagnostic test. The proctor device can be configured to allow the proctor to indicate their interpretation of the results of the user’s medical diagnostic test.

At block 1710, the user of a user device may be prompted to capture information of a medical diagnostic test. The information can include an image, video, audio recording, etc. At block 1720, the user may be prompted to indicate their interpretation of a result of their medical diagnostic test.

At block 1730, a system may receive an image of the medical diagnostic test as captured by the user device. The system may also be configured to receive data indicating the user’s interpretation of the results of the medical diagnostic test. At block 1740, the image of the diagnostic test as captured by the user device may be presented to a proctor. At block 1750, the proctor may be prompted to indicate their interpretation of a result of the medical diagnostic test as shown in the image captured by the user.

At block 1760, the system may receive data indicating the proctor’s interpretation of the result of the user’s medical diagnostic test. At block 1770, the system may perform one or more operations based on the user’s interpretation of the result of the medical diagnostic test and the proctor’s interpretation of the result of the medical diagnostic test.

The method 1700 can advantageously decrease or eliminate handling of the medical diagnostic test results and the time taken for the proctor to interpret the medical diagnostic test results. In this way, in some embodiments, the user may receive their results to the medical diagnostic test in a more timely fashion.

In some embodiments, an AI model or other program, such as the results interpretation algorithm discussed above, can be configured to interpret some or all received test data. An AI model can have a high accuracy for reading and interpreting tests. In some embodiments, the AI model can use multiple frames, possibly at different scales, to synthesis one or more combined frames. Such an approach can help to reduce the effects of compression, poor resolution, etc., which can potentially reveal features (e.g., control lines, test lines, and so forth) that may be imperceptible to a human proctor reviewing a video or individual images received from a user. In some embodiments, the result determined by the AI model can be compared to a proctor reading. If the two agree, no action may be taken. However, if the AI model and human proctor disagree, the proctor can be prompted to reassess the test. In some embodiments, the proctor can be provided with an image that has been enhanced (e.g., cleaned) by the AI model. If the human proctor and AI still disagree, another human proctor can be used to verify the test result. In some embodiments, error rates can be assigned to proctors, and proctors who have error rates greater than a maximum threshold value can be referred for further training, can be dismissed, and so forth.

### Computer Systems

FIG. 18 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 18. The example computer system 1802 is in communication with one or more portable user devices 1815 and/or one or more data sources 1822 via one or more networks 1818. While FIG. 18 illustrates an embodiment of a computing system 1802, it is recognized that the functionality provided for in the components and modules of computer system 1802 may be combined into fewer components and modules, or further separated into additional components and modules.

The user device 1815 can be configured to include one or more medical sensor subsystems as described above.

The computer system 1802 can comprise a module 1814 that carries out the functions, methods, acts, and/or processes described herein (e.g., computing for data received from user devices or medical sensor subsystems of the user devices). The module 1814 is executed on the computer system 1802 by a central processing unit 1806 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 1802 includes one or more processing units (CPU) 1806, which may comprise a microprocessor. The computer system 1802 further includes a physical memory 1810, such as random-access memory (RAM) for temporary storage of information, a read only memory (ROM) for permanent storage of information, and a mass storage device 1804, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 1802 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 1802 includes one or more input/output (I/O) devices and interfaces 1812, such as a keyboard, mouse, touch pad, and printer. The I/O devices and interfaces 1812 can include one or more display devices, such as a monitor, that allows the visual presentation of data to a user. More particularly, a display device provides for the presentation of GUIs as application software data, and multi-media presentations, for example. The I/O devices and interfaces 1812 can also provide a communications interface to various external devices. The computer system 1802 may comprise one or more multi-media devices 1808, such as speakers, video cards, graphics accelerators, and microphones, for example.

The computer system 1802 may run on a variety of computing devices, such as a server, a Windows server, a Structure Query Language server, a Unix Server, a personal computer, a laptop computer, and so forth. In other embodiments, the computer system 1802 may run on a cluster computer system, a mainframe computer system and/or other computing system suitable for controlling and/or communicating with large databases, performing high volume transaction processing, and generating reports from large databases. The computing system 1802 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 1802 illustrated in FIG. 18 is coupled to a network 1818, such as a LAN, WAN, or the Internet via a communication link 1816 (wired, wireless, or a combination thereof). Network 1818 communicates with various computing devices and/or other electronic devices. Network 1818 is communicating with one or more computing systems 1820 and one or more data sources 1822. The module 1814 may access or may be accessed by computing systems 1820 and/or data sources 1822 through a web-enabled user access point. Connections may be a direct physical connection, a virtual connection, and other connection type. The web-enabled user access point may comprise a browser module that uses text, graphics, audio, video, and other media to present data and to allow interaction with data via the network 1818.

Access to the module 1814 of the computer system 1802 by computing systems 1820 and/or by data sources 1822 may be through a web-enabled user access point such as the computing systems’ 1820 or data source’s 1822 personal computer, cellular phone, smartphone, laptop, tablet computer, e-reader device, audio player, or another device capable of connecting to the network 1818. Such a device may have a browser module that is implemented as a module that uses text, graphics, audio, video, and other media to present data and to allow interaction with data via the network 1818.

The output module may be implemented as a combination of an all-points addressable display such as a cathode ray tube (CRT), a liquid crystal display (LCD), a plasma display, or other types and/or combinations of displays. The output module may be implemented to communicate with input devices 1812 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 1802 may comprise a physical or logical connection established between a remote microprocessor and a mainframe host computer for the express purpose of uploading, downloading, or viewing interactive data and databases online in real time. The remote microprocessor may be operated by an entity operating the computer system 1802, including the client server systems or the main server system, an/or may be operated by one or more of the data sources 1822 and/or one or more of the computing systems 1820. In some embodiments, terminal emulation software may be used on the microprocessor for participating in the micro-mainframe link.

In some embodiments, computing systems 1820 who are internal to an entity operating the computer system 1802 may access the module 1814 internally as an application or process run by the CPU 1806.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user’s computer. This data can be stored by a user’s web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 1802 may include one or more internal and/or external data sources (for example, data sources 1822). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 1802 may also access one or more databases 1822. The databases 1822 may be stored in a database or data repository. The computer system 1802 may access the one or more databases 1822 through a network 1818 or may directly access the database or data repository through I/O devices and interfaces 1812. The data repository storing the one or more databases 1822 may reside within the computer system 1802.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open- ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A method for image analysis of a medical diagnostic test, the method comprising:
receiving, by an image verification server, specifications of a camera of a user device;
comparing, by the image verification server, the specifications to a database of specifications to determine if the specifications are sufficient for use in the medical diagnostic test;
if the image verification server determines the specifications are sufficient for use in the medical diagnostic test, prompting, by a telehealth platform in communication with the image verification server, a user to capture an image of a reference using the camera of the user device;
receiving, by the image verification server, the image of the reference;
automatically processing, by the image verification server, the image of the reference using a calibration model to produce a processed image, wherein the calibration model is based on the specifications;
determining, by the image verification server, whether a quality of the processed image is above a predetermined threshold;
if the processed image does not have a quality above the predetermined threshold, automatically updating, by the image verification server, the calibration model, processing, by the image verification server, the processed image using the calibration model, and determining, by the image verification server, whether the quality of the processed image is above the predetermined threshold;
if the processed image has a quality above the predetermined threshold, prompting, by the telehealth platform, the user to capture an image of a diagnostic test result;
receiving, by the image verification server, the image of the diagnostic test result;
processing, by the image verification server, the image of the diagnostic test result using the calibration model to produce a processed test image;
transmitting, by the image verification server, the processed test image to the telehealth platform; and
analyzing, by the telehealth platform, the processed test image to determine if the diagnostic test result is positive or negative.

2. The method of claim 1, wherein the reference is a reference card comprising a unique code, graduated color strips, a plurality of color reference blocks, and a plurality of fiducials.

3. The method of claim 2, wherein the quality of the processed image is based on one or more of a resolution, a sharpness, a brightness, a noise, a dynamic range, a contrast, a saturation, and/or a white balance.

4. The method of claim 1, wherein the image verification server generates an image quality score of the quality of the processed image.

5. The method of claim 1, wherein the user device comprises a medical sensor subsystem, the medical sensor subsystem comprising:
the camera;
an image signal processor;
an autofocus driver; and
a circuit.

6. The method of claim 5, wherein the medical sensor subsystem meets guidelines or regulations of a regulatory body.

7. The method of claim 5, wherein the camera is separated from the image signal processor, the autofocus driver, and the circuit via a shield.

8. The method of claim 5, wherein the image verification server is configured to:
Receive a plurality of images from a plurality of user devices comprising the medical sensor subsystem; and
update the calibration model based on the quality of the plurality of images.

9. The method of claim 1, wherein the image verification server is a remote image verification server separate from the user device.

10. The method of claim 1, wherein the image verification server transmits an indication of the determination whether the quality of the processed image is above the predetermined threshold to the user device and/or a partner device, wherein the partner device is a device of a proctor.

11. A method for self-guided testing, the method comprising:
receiving, by a telehealth platform, a first video of a user performing one or more self-guided steps of a first phase of a medical diagnostic testing procedure, wherein the first video comprises a first frame rate;
receiving, by the telehealth platform, a second video of the user performing one or more self-guided steps of a second phase of a of the medical diagnostic testing procedure, wherein the second video comprises a second frame rate, and wherein the second frame rate is higher than the first frame rate;
transmitting, by the telehealth platform, the first video and the second video to a proctor device;
displaying, by the telehealth platform, the first video and the second video to a proctor via the proctor device;
receiving, by the telehealth platform, an input from the proctor, the input indicating whether the user performed the first phase and the second phase in compliance with the medical diagnostic testing procedure;
based on the input from the proctor, selecting, by the telehealth platform, a third phase of the medical diagnostic testing procedure, wherein the third phase comprises one or more steps; and
prompting, by the telehealth platform, the user to perform the one or more steps of the third phase.

12. The method of claim 11, wherein the first phase comprises the user setting up testing materials or collecting a sample.

13. The method of claim 11, wherein the second phase comprises the user waiting for a predetermined amount of time.

14. The method of claim 11, wherein the input indicates the user performed the first phase and the second phase in compliance with the medical diagnostic testing procedure, and the one or more steps of the third phase comprise a test result interpretation procedure.

15. The method of claim 14, wherein the test result interpretation procedure comprises:
receiving, by the telehealth platform, an image of a test result and an interpretation of the test result input by the user;
displaying, by the telehealth platform, the image of the test result to the proctor via the proctor device;
receiving, by the telehealth platform, a second interpretation of the test result input by the proctor; and
analyzing, by the telehealth platform, the interpretation and the second interpretation to determine the test result of the medical diagnostic testing procedure, wherein said analyzing comprises comparing the interpretation and the second interpretation to determine if the interpretation and the second interpretation indicate a same test result.

16. The method of claim 11, wherein the input indicates the user performed the first phase and/or the second phase not in compliance with the medical diagnostic testing procedure, and the one or more steps of the third phase comprise the telehealth platform connecting the user with the proctor for assistance with the medical diagnostic procedure.

17. A method of interpreting test results of a medical diagnostic test, the method comprising:
receiving, by a telehealth platform, an image of a test result and an interpretation of the test result input by a user;
displaying, by the telehealth platform, the image of the test result to a proctor via a proctor device;
receiving, by the telehealth platform, a second interpretation of the test result input by the proctor; and
analyzing, by the telehealth platform, the interpretation and the second interpretation to determine a test outcome of the medical diagnostic test, wherein said analyzing comprises comparing the interpretation and the second interpretation to determine if the interpretation and the second interpretation indicate a same test result.

18. The method of claim 17, wherein if the interpretation and the second interpretation indicate the same test result, the test outcome is the same test result.

19. The method of claim 17, wherein if the interpretation and the second interpretation do not indicate the same test result, the telehealth platform is configured to:
analyze the image of the test result, via a results interpretation algorithm, to determine a third interpretation of the test result;
display the image of the test result to a second proctor via a second proctor device;
receive a fourth interpretation of the test result input by the second proctor;
analyze the third interpretation and the fourth interpretation to determine the test outcome of the medical diagnostic testing procedure, by comparing the third interpretation and the fourth interpretation to determine if the third interpretation and the fourth interpretation indicate a same test result,
if the third interpretation and the fourth interpretation indicate a same test result, the test outcome is the same result, and
if the third interpretation and the fourth interpretation do not indicate a same test result, the test outcome is invalid, and the telehealth platform is configured to prompt the user to retake the medical diagnostic test.

20. The method of claim 17, wherein if the interpretation and the second interpretation do not indicate the same test result, the telehealth platform is configured to:
connect the user to a representative, wherein the representative reviews the test result with the user to guide the user to modify the interpretation to the same test result as the second interpretation, and
if the user modifies the interpretation to the same test result as the second interpretation, the test outcome is the same test result,
if the user does not modify the interpretation to the same test result as the second interpretations, the representative indicates the test outcome is invalid.
